(19) United States
(12) Patent Application Publication
(10) Pub. No.: US 2006/0270919 A1
(43) Pub. Date: Nov. 30, 2006

(54) Biomarkers sensing

(75) Inventor: Mary K. Brenner, Plymouth, MN (US)

Correspondence Address: KINNEY & LANGE, P.A., THE KINNEY & LANGE BUILDING, 312 SOUTH THIRD STREET, MINNEAPOLIS, MN, 55415-1002, US

(73) Assignee: Mytek, LLC, Maple Grove, MN (US)

(21) Appl. No.: 11/430,436

(22) Filed: May 9, 2006

Related U.S. Application Data
(60) Provisional application No. 60/679,878, filed on May 11, 2005.

(51) Int. Cl. A61B 5/00

(52) U.S. Cl. 600/310; 600/317

(57) ABSTRACT

A method of, and system for, assaying for selected constituents in liquid mixtures confined by corresponding containing structures having relatively small electromagnetic absorption in at least one transmission wavelength range for transmissions of electromagnetic radiation therethrough, including in vivo assaying for a presence of selected constituents in bloodstreams in circulatory system passageways in mammalian bodies having relatively small electromagnetic radiation absorption in at least one transmission wavelength range for transmissions of electromagnetic radiation between such passageways and outer surfaces of corresponding bodily skin, based on, in the bloodstream example, introducing in a bloodstream a probe comprising a binding base capable of binding to at least one of the selected constituents and of also concurrently conjugating to two different fluorophores of which one has an emission spectra peak distribution in the transmission wavelength range that overlaps an absorption spectra peak distribution of that one remaining. Radiate at least some of the passageways from at least one location on the outer surfaces of the corresponding skin with electromagnetic radiation of wavelengths in the transmission wavelength range and also in an absorption spectra range of that fluorophore having an overlapping emission spectra peak distribution. Detect electromagnetic radiation at least at one location on the outer surfaces of the corresponding skin that has been transmitted from the evaluative radiated passageways of wavelengths in the transmission wavelength range and also in an emission spectra range of that fluorophore having an overlapping absorption spectra peak distribution wherein the emission spectra range differs from the overlapping emission spectra peak distribution.

### CROSS-REFERENCE TO RELATED APPLICATION(S)

This application claims the benefit of Provisional Application No. 60/679,878 filed May 11, 2005 for “BIOMARKERS SENSING”.

### BACKGROUND OF THE INVENTION

The present invention relates to monitoring biological entities and chemicals in the bloodstreams of biological organisms and, more particularly, to sensing such elements using electromagnetic radiation based methods and equipment.

Two trends occurring within biological and medical diagnostics are subject or patient continuous monitoring, and point-of-care patient diagnostics. These have been developed in response to a) the need for more rapid information return on the biological or health status of a subject or a patient, and to b) the desire to provide real-time or near-real-time information in the direct management of a medical condition. Specific examples of continuous monitoring providing real-time, or near real-time, information feedback include the measurement of the oxygenation of blood with pulse oximeters, and motor activity monitors which provide feedback to devices being concurrently used by the subject or patient such as pacemakers. There is recently much activity in the developing of bloodstream glucose monitors to provide real-time feedback to insulin pumps.

Point-of-care instruments have also emerged over the last years. These devices are smaller than typical diagnostic laboratory instruments, and are located near where patient care is provided. Some devices measure blood chemistry such as its content of sodium, potassium, and some measure partial pressures of gases in the blood. Others measure blood for contents of drugs of abuse. Another category of devices are those which measure cardiac biomarkers, i.e. proteins that are indicators of damage to the heart muscle. Generally these devices have required blood to be drawn and added to a disposable cartridge, and the cartridge is then inserted into an analyzing instrument which quantifies the measurand of interest.

In the absence of these alternatives, testing is done by taking a blood or tissue sample and sending it to a central laboratory, either a central laboratory within a hospital or at a large testing organization facility that contracts to do testing for healthcare providers and healthcare facilities. Within a hospital, the information return on a test can take an hour or more: the sample must be collected, transported to the laboratory, tested and the results communicated back to the patient location. Emergency department personnel often find the delays inherent in this process especially frustrating.

Regardless of whether the diagnostic test is done in the central laboratory or on a “Point-of-Care” instrument, this testing is usually done infrequently, and requires the time and attention of medical caregivers, i.e. nurses, emergency room personnel, etc. Results and background concentrations can vary from person to person, and trends may not be observable unless measurements are being made very frequently thereby adding to such time and attention requirements.

Cardiac monitoring encompasses a variety of techniques and provides an example of how the emergence of monitoring of biomarker bloodstream proteins is being accomplished today, as well as the potential of biomarker monitoring techniques for the future. Typically in the past, and still today, continuous electrocardiographic (EKG) monitoring is ordered for patients or other subjects with known or suspected myocardial infarction (or heart attack). Continuous electrocardiographic (EKG) and hemodynamic monitoring of patients with acute coronary syndromes and congestive heart failure is the current standard of care. Standard 12-lead EKGs remain the most commonly employed diagnostic tool in clinical cardiology. Wireless transmission of the EKG signal to a central hospital telemetry unit allows the patient to undergo procedures or be transferred between hospital units while being monitored. Wireless transmission also provides continuous information for patients at high risk for arrhythmias.

Nevertheless, EKGs are less than ideal in their diagnosis of acute myocardial infarction. Many patients are retained in the hospital unnecessarily because of the uncertainty as to whether they have suffered a myocardial infarction, while 1 to 2% of actual cases still go undetected. However, the more recent movement toward detecting various cardiac biomarkers, and the associated development of corresponding test instruments, has helped to remedy the problem. Cardiac biomarkers are proteins that are released into the blood when damage is done to the heart muscle, and hence their presence in blood is an indicator that such damage has occurred.

Another example of continuous monitoring in cardiology is the continuous invasive hemodynamic monitoring using Swan-Ganz catheters for congestive heart failure patients. This is done to provide diagnosis, but also to guide therapy. Continuing to monitor the status of congestive heart failure patients following their release from the hospital is desirable, and tests based upon cardiac biomarkers have been developed to do this. However, this monitoring is currently accomplished by fairly infrequent trips to doctors' offices for blood draws and tests.

Historically, lactase dehydrogenase and creatine kinase were the first biomarkers found for indicating cardiac necrosis. The monitoring of those substances was subsequently replaced by the monitoring of the creatine-kinase MB isoenzyme (CK-MB), which in turn has been supplemented by the monitoring of the cardiac troponins. CK-MB is still used in cases of early post-myocardial infarction ischemia or re-infarction. Myoglobin, due to its early rise, is considered a hyperacute marker and is recommended in a multiple biomarkers diagnostic strategy of monitoring cardiac troponin I (cTnI) or T (cTnT) and myoglobin.

The analytical tools used for the detection of these biomarkers in peripheral blood samples have also evolved from radio-immune techniques to the current enzyme immunoassay-based large clinical laboratory analytical platforms. In an effort to decrease the turn-around time from originally 90 minutes to an average of 15 minutes in most commercially assays available today, various strategies have been employed, including the development of point-of-care devices which combine good analytical precision, rapid information return, portability and the potential for use at bedsides or at small institutions where large clinical laboratory platforms are not financially viable options.

A release pattern for cTnI, CK-MB, and myoglobin during an acute coronary syndrome is illustrated in the graph shown in FIG. 1 with plots typical of that provided by cardiac biomarker test manufacturers. A first plot, 10, is for myoglobin, a second plot, 11, is for troponin-1, and the a final plot, 12, is provided for CK-MB. Results, however, vary among patients. Myoglobin peaks approximately 2 hours after the onset of typical angina and allows for the identification of myocardial infarction. However, its tissue specificity is poor (i.e. it can be released in response to damage to other non-cardiac types of muscle) and its diagnostic time window narrow, which leads to inconsistent performance. CK-MB rises quickly and decays within 36 to 48 hours to normal concentrations. CK-MB has been replaced by cardiac troponins for the diagnosis of acute coronary syndromes but is still used for the detection of early post-infarction ischemia and reinfarction. CK-MB is abundant in normal skeletal muscles and lacks the absolute cardiac tissue specificity of cardiac troponins. Cardiac troponins are not normally present in serum unless cardiac cell necrosis has occurred, and thus are more specific than CK-MB. The concentrations remain elevated for up to 14 days after myocardial injury, thereby also allowing the retrospective diagnosis of an acute coronary event in patients who delay seeking medical care. Multiple biomarker diagnostic strategies have been recommended and point-of-care systems have been developed for improved results.

FIG. 1 also illustrates some of the issues that remain in the use of cardiac biomarkers for the diagnosis of myocardial infarction. Even with point-of-care instruments, the tests are done infrequently, perhaps every 8 hours. When a patient arrives at a hospital with chest pain, health care providers often do not know when the damage might have first occurred, and so do not know where on the curves of FIG. 1 the patient's condition currently lies. Some patients have a background concentration of one or more of the biomarkers. Furthermore, it is important, but can be difficult, to detect the occurrence of a second or third heart attack taking place during the first day or two after the initial attack. The ability to track the concentrations of the cardiac biomarkers in the blood on a more or less continuous basis would enhance the diagnosis, and so lead to more appropriate treatment of heart disease and damage.

The continuous monitoring of a component of a patient's or subject's blood implies the need to measure that component “in vivo”, that is, in the body of that person. However, most of the measurands of interest are present in blood in quite small concentrations. The oxygen content of the blood is an exception. The oxygen concentration in the blood is sufficiently large that it can be measured (although still with some limitations) through directing optical radiation into the blood followed by measuring the absorption and scattering through a resulting optical signal at two different specific wavelengths. However, the difficulties that have existed in developing noninvasive sensors for glucose illustrate the problem in the more typical blood concentration situation.

Glucose is more plentiful in the blood than most of the other measurands of interest (in concentrations of milligrams/milliliter), and yet a reliable completely noninvasive in vivo measurement means has yet to be demonstrated. As an example, if relying on optical scattering or absorption techniques to determine the amount of glucose present, the presence of other substances, whose concentrations are usually not well known, affects the signal representing glucose that one is trying to measure. Other substances of interest, such as the cardiac proteins, are present at even lower concentrations, i.e. micrograms/milliliter or nanograms/milliliter. One of the optical techniques that is applied “in vitro” (literally, “in glass”, or outside the body) and which provides the best sensitivity is based on the occurrence of matter fluorescence. A possibility here for in vivo measurements is to make use of auto-fluorescence, or the natural fluorescence of tissues, but that poses two issues: a) it is difficult to distinguish the fluorescence of one type of tissue from another, and 2) tissues fluoresce in the UV and visible wavelengths that are absorbed by the tissue itself, and hence it is difficult to transmit the signal outside the body. Therefore, the addition of fluorescing dyes will be required to make this approach feasible in many instances.

One of the key issues in contemplating an in vivo assay is the ability to generate a fluorescent signal which is specific to the measurand of interest. Currently many in vitro fluorescent assays for proteins implement a two step process: 1) an antibody conjugated with a dye reacts with the protein of interest (which functions as the antigen, and 2) the antigen reacts with a second antibody attached to a substrate, termed the “capture” antibody. The unattached antibodies are then rinsed away. However, in the case of an in vivo assay it isn't desirable, and may not even be feasible, to implant a substrate for the capture, and it is not possible to flush away the unreacted fluorescent antibodies. Thus, there is a desire for a system that can provide in vivo assays of selected blood components.

### BRIEF SUMMARY OF THE INVENTION

The present invention provides a method of assaying for selected constituents in liquid mixtures confined by corresponding containing structures having relatively small electromagnetic absorption in at least one transmission wavelength range for transmissions of electromagnetic radiation therethrough, including in vivo assaying for a presence of selected constituents in bloodstreams in circulatory system passageways in mammalian bodies having relatively small electromagnetic radiation absorption in at least one transmission wavelength range for transmissions of electromagnetic radiation between such passageways and outer surfaces of corresponding bodily skin, based on in the bloodstream example introducing in a bloodstream a probe comprising a binding base capable of binding to at least one of the selected constituents and of also concurrently conjugating to two different fluorophores of which one has an emission spectra peak distribution in the transmission wavelength range that overlaps an absorption spectra peak distribution of that one remaining. Thereafter, the method continues by radiating at least some of the passageways, thereby forming evaluative radiated passageways, from at least one location on the outer surfaces of the corresponding skin with evaluation electromagnetic radiation of wavelengths in the transmission wavelength range and also in an absorption spectra range of that fluorophore having an overlapping emission spectra peak distribution. In conjunction with such radiating, also detecting electromagnetic radiation at least at one location on the outer surfaces of the corresponding skin that has been transmitted from the evaluative radiated passageways of wavelengths in the transmission wavelength range and also in an emission spectra range of that fluorophore having an overlapping absorption spectra peak distribution wherein the emission spectra range differs from the overlapping emission spectra peak distribution.

Such methods can be used with a body supported bloodstream biomarker evaluation system for such assaying with this system being provided on a substrate supporting a first electromagnetic radiation emitter capable of emitting evaluation electromagnetic radiation of wavelengths in the transmission wavelength range and also capable of emitting evaluation electromagnetic radiation of wavelengths in the absorption spectra range of that fluorophore having an overlapping emission spectra peak distribution. Further supported on the substrate is a first electromagnetic radiation detector capable of detecting electromagnetic radiation of wavelengths in the transmission wavelength range and also in the emission spectra range of that fluorophore having an overlapping absorption spectra peak distribution.

### BRIEF DESCRIPTION OF THE DRAWINGS

FIG. 1 shows a graph having plots representing the time dependence of cardiac biomarkers following acute myocardial infarctions,

FIG. 2 shows a graphical schematic representation of the FRET process,

FIG. 3 shows a graph with plots of absorption coefficients versus wavelengths for various biological tissue types illustrating a “therapeutic window” between 600 and 1400 nm,

FIG. 4A shows a top view of a representation of an embodiment of the biomarker sensor of the present invention,

FIG. 4B shows a top view of an alternative embodiment of the present invention,

FIG. 4C shows a top view of a further alternative embodiment of the present invention,

FIG. 4D shows a top view of a further alternative embodiment of the present invention,

FIG. 5A shows a side view in cross section of an abbreviated version of the embodiment shown in FIG. 4A,

FIG. 5B shows a side view in cross section in an abbreviated version of an alternative to the embodiment shown in FIG. 4A,

FIG. 6A shows a side view in cross section in an abbreviated version of a further alternative to the embodiment shown in FIG. 4A,

FIG. 6B shows a side view in cross section in an abbreviated version of a further alternative to the embodiment shown in FIG. 4A,

FIG. 7A shows a layer diagram of a resonant cavity photodetector used in the embodiment shown in FIG. 4A,

FIG. 7B shows a layer diagram of a p-i-n photodiode with an external filter used in the embodiment shown in FIG. 4A,

FIG. 8 shows a graph with plots of absorption of radiation versus wavelengths in oxygenated and deoxygenated blood,

FIG. 9 shows a schematic arrangement illustrating the principle of distance measurement by triangulation,

FIGS. 10A and 10B show graphs with plots of absorption and emission spectra of selected dyes, the emissions of excitation VCSELs versus wavelengths, and the absorptions of photodetectors versus wavelengths,

FIG. 11A shows a graphs with plots of the emissions of representative excitation LEDs and VCSELs versus wavelengths,

FIGS. 11B and 11C show graphs with plots of the responsivities of selected representative photodetectors versus wavelengths, and

FIG. 12 shows graphs of fluorescence intensity versus time representative of dyes.

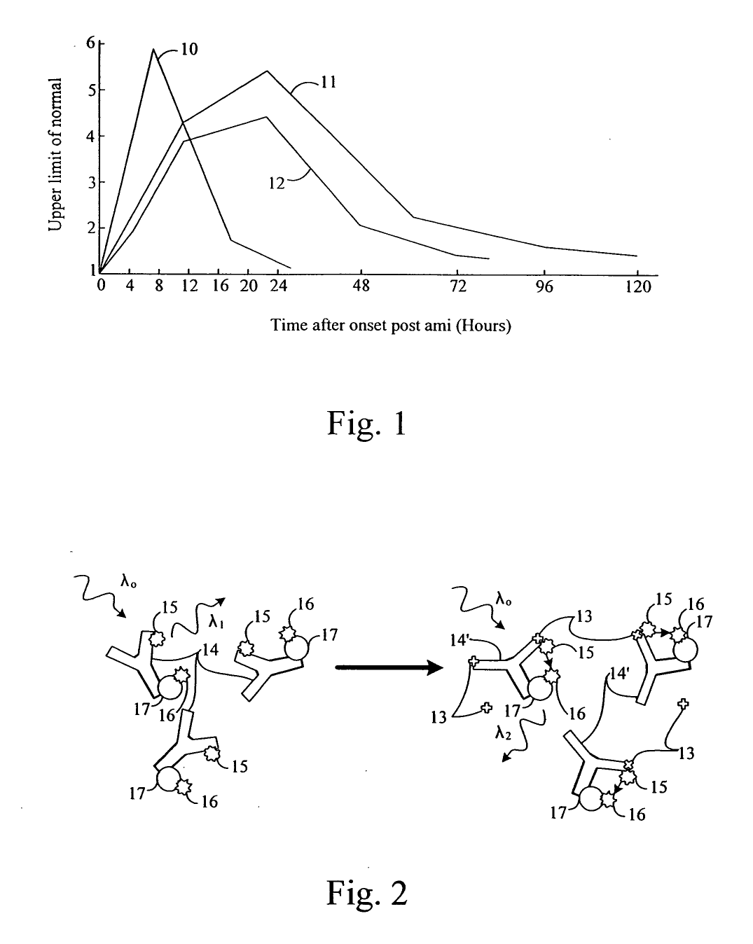

### DETAILED DESCRIPTION

Rather than the current technique being used for in vivo fluorescent assays for blood proteins described above, the technique of fluorescence resonant energy transfer (FRET) is more suitable for monitoring patient's or subject's blood. The FRET technique has recently been applied to the detection of cardiac troponins in vitro. In this instance the fluorophores (dyes) were conjugated to a troponin I antibody-Protein G complex and a Troponin T antibody Protein A complex, and were allowed to react in solution with a sample containing troponin. All of the reagents used in the assay are available from commercial sources. A change in the fluorescence signal and detection of troponin was demonstrated all without the use of a capture substrate or the need to flush away the unreacted dye conjugated proteins as described below.

FIG. 2 schematically illustrates the FRET technique in showing the effect of antigens, 13, on antibodies, 14, to an arm of which a dye, 15, or fluorophore 1, is attached and to another arm of which another dye, 16, or fluorophore 2, is attached through an extra Protein A or G, 17, directly bound to that antibody second arm. Proteins 17 serve to keep fluorophores 1 and 2, or the dyes, sufficiently separated as positioned with respect to corresponding antibody 14. Thus, these dyes, capable after being stimulated, or excited, of emitting electromagnetic radiation at two different wavelengths, are initially conjugated to a corresponding antibody 14 as shown to the left of the arrow of elapsed time in FIG. 2. In the absence of antigens 13, the excitation incoming radiation of wavelength λₒ excites dye 15 to result in that dye emitting light at wavelength λ₁. However, when in time a protein (serving as antigen 13) of interest reacts with, or binds to, an antibody 14, as shown to the right of the bolded horizontal elapsed time arrow in the figure, the configuration of that antibody changes (and so designated 14′ on the right side of the elapsed time arrow in FIG. 2), bringing the two dyes, or fluorophores 1 and 2, closer together. Then a nonradiative energy transfer occurs from fluorophore 1 to fluorophore 2 as indicated by the short arrow between them such that, when the exciting radiation of wavelength λₒ is incident upon the molecule, light of wavelength λ₂ is emitted. In this manner, an effective probe is developed that is specific to the protein of interest, without requiring a substrate upon which the reaction can take place. The signals of the reacted and unreacted antibodies are separated in wavelength to thereby be separately detectable, so it is not necessary to flush away the unreacted material.

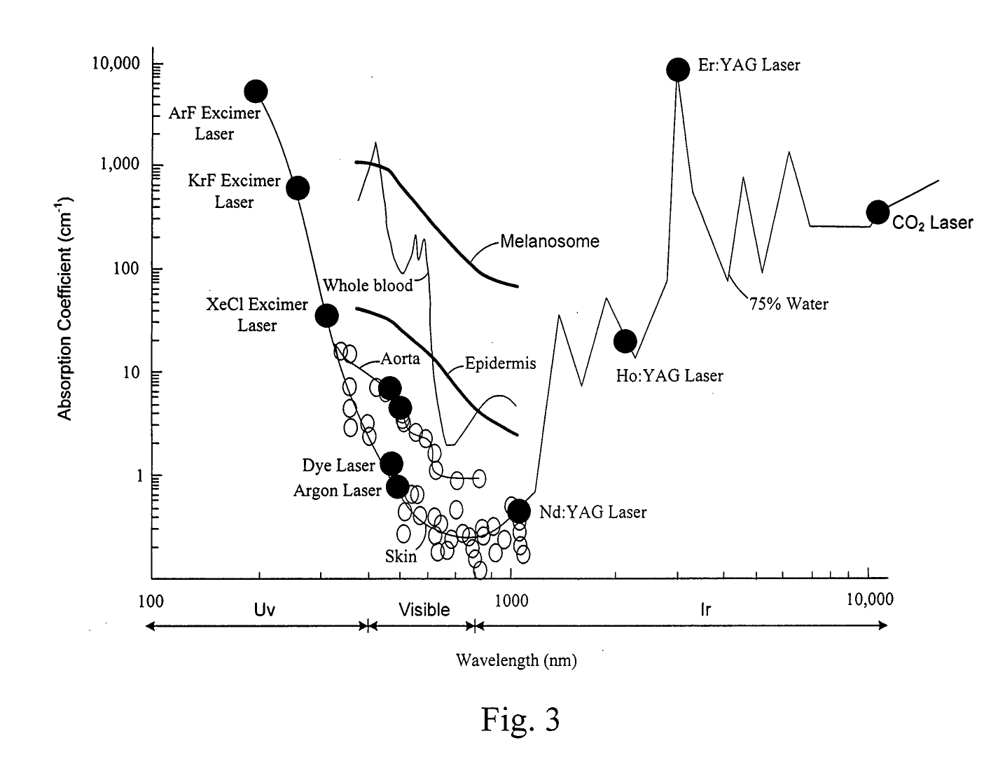

Such dyes must be capable of operating in the wavelength range in which relatively little absorption occurs in the human body being assayed or monitored so that this range can constitute an electromagnetic radiation transmission window for the body. This is termed a “therapeutic window” and is commonly understood to range from around 600 nm to 1400 nm as is indicated in the plot in the graph of FIG. 3.

Dyes are now available in which the absorption and emission occurs in the red and near infrared portions of the electromagnetic radiation spectrum which is within this therapeutic window. For instance, the cyanine class of dyes are available from the 450 nm to 900 nm range, and exhibit high molar extinction coefficients (>150,000 M⁻¹ cm⁻¹) and good fluorescence quantum yields (up to 50%). The Alexa Fluor dyes from Molecular Probes (Eugene, Oreg.) and the CyDye™ series from Amersham—Pharmacia Biotechnology (Piscataway, N.J.) are two commercially available series of dyes that encompass the wavelength range of interest. Dyes absorbing and emitting in the red and near-IR have been applied to a whole blood immunoassay in the Triage System (Biosite Incorporated, San Diego, Calif.). In that system, the excitation energy of the donor dye is 670 nm, while the emission light of the acceptor dye is 760 nm.

A rough calculation indicates that something on the order of a 0.1 cc quantity of conjugated antibody would be required to be introduced into the body being assayed or monitored. Minimum sensitivities in currently available assays are around 0.1 μg/L for cTnT, for instance, while the maximum concentrations observed of cTnI in one study were less than 100 μg/L. Typically, there are approximately 5 L of blood in a human body, and so the maximum concentration in the body would be less than 500 μg. Assuming the concentration of conjugated antibodies required is similar, and that the concentration is a typical 5 mg/mL, then the volume of the injection would be approximately 0.1 mL or 0.1 cc. While this is an approximate estimate it is clearly in the range of what is feasible.

The dye conjugated antibodies to be introduced into a human body for such assays or monitoring must not pose any toxicity concerns. However, the concept of using dye conjugated antibodies for in vivo imaging (as opposed to the continuous monitoring) is being very actively pursued by many researchers, with the greatest activity being directed to the imaging of cancer related tissues. The probes being used include dyes attached to small molecules, such as receptor ligands or enzyme substrates, as well as higher molecular weight probes such as monoclonal antibodies or recombinant proteins. Probes have been reported to have been detected in vivo in nanomolar concentrations with no apparent toxicity issues.

A compact system for fluorescence based immunoassays also requires the availability of optical sources and detectors appropriate for the task. Significant performance improvements and cost reductions in optoelectronic components have occurred over the last decade which make feasible the development of wearable or portable sensors that enable continuous monitoring.

Electromagnetic radiation emitters available during the early years of the development of spectroscopic testing were bulky such as broad spectrum lamps, or large, gas lasers. Compact semiconductor electromagnetic radiation emitting sources now available enable the fabrication of compact instruments which are more convenient. Thus, semiconductor light emitting diodes (LEDs) are small and inexpensive but they have broad spectral peaks and are relatively slow for use in time-resolved measurements.

Semiconductor laser diodes for use in fiber optic based arrangements, such as in optical fiber networks for long distance telephone communication, were developed at wavelengths of 850 nm and 1310 nm because of the relatively low absorption transmission windows at those wavelengths in the optical fibers used, but were initially relatively expensive. With the advent of the sales of compact disk (CD) players in large numbers, very low cost laser diodes became available initially at the 780 nm wavelength and now at 635 and 650 nm for digital video disk (DVD) players.

In the early 1990s, a new type of semiconductor laser diode became available that provided the advantages of both LEDs and semiconductor laser diodes. This device, called a Vertical Cavity Surface Emitting Laser, or VCSEL, has a vertically emitting structure (like the LED) that enables a wide variety of device packaging alternatives. However, it also has high electrical power efficiency (10-30%), a narrow symmetrical output beam (<15° at full width half maximum), a narrow wavelength spectrum (<0.1 nm), and very fast rise and fall times (<100 psec) that are typical of a laser diode. Such laser diodes were initially developed to emit at 850 nm for use with local area fiber optical networks, but such devices with emission wavelengths ranging from 650 nm to 1550 nm have been demonstrated.

Similarly, high responsivity photodetectors have become widely available that are capable of detecting electromagnetic radiation in the same wavelength range. Such photodetectors can be combined either with external radiation filters, or monolithically integrated radiation filters transmitting electromagnetic radiation in relatively narrow bands of wavelengths to thereby improve the noise characteristics of sensed radiation signals.

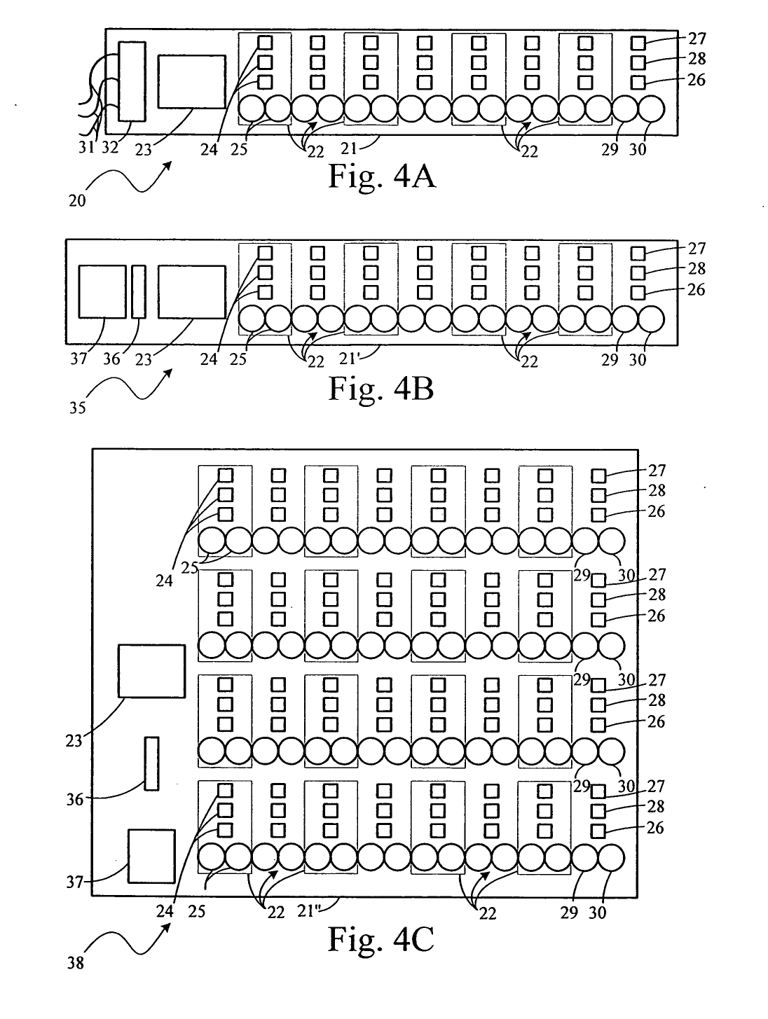

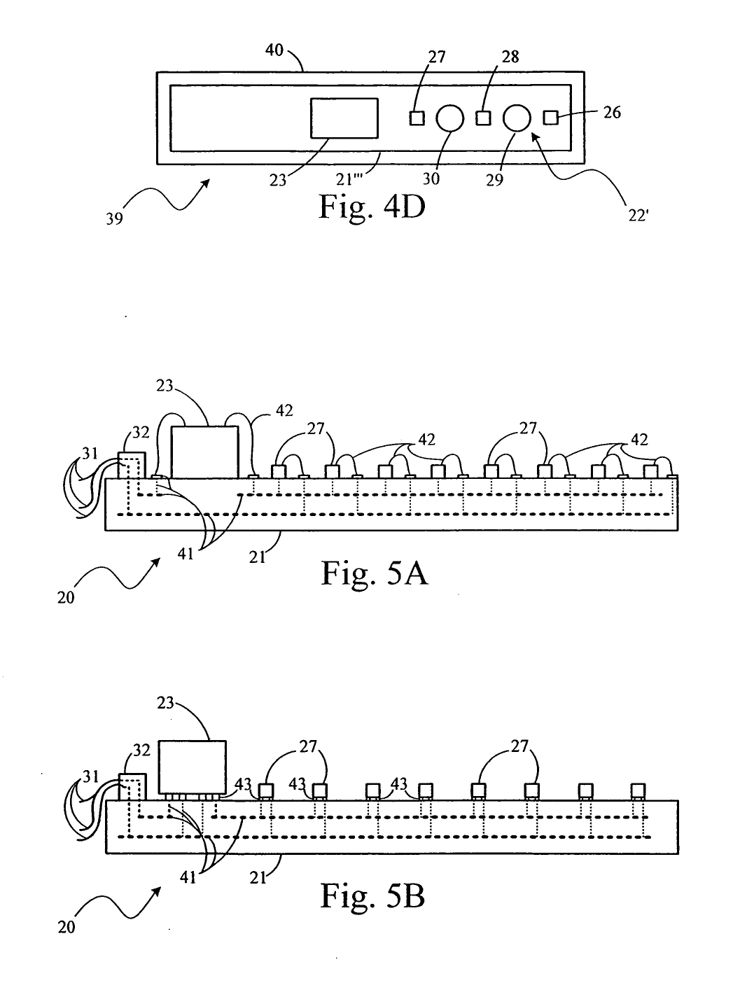

An example of implementing a FRET based technique for immunoassays and monitoring of selected blood components is shown in FIG. 4A which provides a top view of a schematic representation of a wearable assaying system of the present invention which includes optoelectronic components serving as optical sources and detectors, an integrated circuit to provide control and signal processing capabilities, and an electrical power source. This assaying system is typically worn on an arm of a human subject or patient who has had suitable fluorescent dyes added into his or her bloodstream to conjugate with antibodies or enzymes that react with the protein, or chemical, in that bloodstream that is of interest to be monitored by this bloodstream evaluation system.

This bloodstream assaying, or bloodstream biomarker sensing device, 20, includes a flexible or rigid substrate, 21, (chosen as indicated below) made of a standard electronic packaging or board material such as Kapton, which contains conductive traces serving as interconnection wiring for suitably interconnecting the devices mounted on it. Optoelectronic device clusters, 22, of electromagnetic radiation emitters and detectors, every other one being delineated for clarity by dashed line rectangles thereabout, are positioned across substrate 21 with the position center to position center spacing of adjacent clusters being approximately 0.5 mm. Also located on the substrate is a control and signal processing integrated circuit chip, 23, containing a processor that provides operating signals for the optical emitters and provides selected processing of the detected radiation signals received thereby from the corresponding photodetectors.

Each cluster 22 of optoelectronic devices has therein three radiation emitters, 24, and two radiation photodetectors, 25, for implementing FRET based assaying or monitoring of the user. The emission wavelengths of all three emitters are chosen to be within a wavelength range extending from 600 nm to 1400 nm. One of emitters 24 would be an emitter, 26, that emits in the red portion of the electromagnetic spectrum, i.e. at approximately 660 nm, a second one thereof would be an emitter, 27, that emits in the far red portion, i.e. at approximately 680 nm, and a third one would be an emitter, 28, that emits in the near infrared portion, i.e. at approximately 910 nm. All three of emitters 24 are typically VCSELs due to their high power efficiency and narrow spectrum. However, substitute sources can be substituted such as narrowband filtered LEDs or conventional edge-emitting lasers which might be chosen as being sufficient for the purpose to thereby reduce costs.

The two photodetectors 25 have high responsivity in the radiation wavelength ranges matching the emission wavelength ranges of emitters 24. One of these photodetectors, 29, is a narrowband detector to detect in a narrow wavelength range that corresponds to the induced emission wavelength λ₂ of the second dye in a protein bound antibody conjugated to the two dyes as described above with the FRET procedure. The second of these photodetectors, 30, is a broadband detector to detect in a wavelength range that includes the range of 650 nm to 920 nm. Broadband detector 30 is a common, well known Si or GaAs semiconductor material device such as a p-i-n photodiode or metal-semiconductor-metal (MSM) photodetector. Narrowband detector 29, in contrast, is a custom resonant cavity photodetector to allow obtaining the narrowband sensitivity, and will be described in more detail below. A third photodetector could be included for use in further reducing noise associated with the fluorescence signal. This would also be a narrowband detector like 29 and its wavelength detection range would be chosen to match the emission wavelength λ₁ of the first dye described above in the FRET procedure.

A connecting cable, 31, can be soldered to portion of the conductive traces circuit interconnections, or alternatively connected via an electrical connector, 32, attached to substrate 21 to provide access to the radiation representation signal produced by the sensor. Cable 31 will also provide electrical power to the device.

A schematic representation of a further sensing device, 35, is shown in the top view of FIG. 4B that is similar to that shown in FIG. 4A except for a changed dimension substrate, 21′, and the omission of connecting cable 31 and connector 32. In sensing device 35, a radio frequency (RF) communication integrated circuit chip, 36, is added to the larger substrate to receive control communication signals at chip 36 from a corresponding broadcasting source thereof and to provide communication signals containing sensed data from chip 36 to a corresponding receiver. Chip 36 receives and provides communication signals in accordance with some standard communication protocol such as the one commonly known as Bluetooth. A battery, 37, is also included in device 35 to provide electrical power for the various electronic circuits and devices operating therein. This can be a low profile battery such as those typically used in calculators or cameras.

A schematic representation of another sensing device, 38, is shown in the top view of FIG. 4C that is similar to that shown in FIG. 4B except again for a changed dimension substrate, 21″, to permit having optoelectronic clusters 22 be incorporated in a two-dimensional array thereof. Such an array with its added clusters is beneficial in allowing the sensor to choose among more measurement locations to perhaps allow control and signal processing integrated circuit chip 23, or the corresponding broadcasting source and receiver arrangement, to find among the user body locations covered by the array an optimal location in some sense for making the desired blood element measurements.

FIG. 4D represents in a top view yet another alternative schematic representation configuration for a sensing device, 39, that incorporates supported on a further changed dimension substrate, 21′″, but a single, modified optoelectronic cluster, 22′, again having therein emitters 26, 27 and 28 along with photodetectors 29 and 30 as well as incorporating integrated circuit chip 23. In this configuration, substrate 21′″ supporting the optoelectronic cluster 22′ chips and integrated circuit chip 23 is installed in a housing, 40, that can be hermetically sealed. The arrangement will be described in more detail below.

FIGS. 5A and 5B show an abbreviated side view version in cross section of device 20 (which vary little from similar cross sectional abbreviated side view versions of sensing devices 35, 38 and 39 not provided here). Sensing device 20 can be made more flexible, and therefore more comfortable for a using patient or subject, by choosing substrate 21 (and substrates 21′, 21″ and 21′″) to be a flexible Kapton sheet patterned with surface and buried copper traces, 41, forming the electrical interconnections between integrated circuit chip 23 and emitters 26, 27 and 28 of optoelectronic devices clusters 22 (and 22′), and between integrated circuit chip 23 and photodetectors 29 and 30 of those clusters.

Alternatively, such substrates can be chosen to be a rigid Kapton or other material circuit board, or a rigid electrical or plastic submount. These last configurations might be chosen if the sensing device is to be reused multiple times and therefore needs to be more rugged.

In the side cross section view of FIG. 5A, the various chips in sensing device 20 are mounted on substrate 21 and are electrically connected to interconnection traces 41 of substrate 21 through aluminum or gold bond wires, 42, extending from pads on those chips to the portions of traces 41 exposed at the upper surface of substrate 21. Although connecting cable 31 can alternatively be soldered directly to portions of traces 41 exposed at the upper surface of substrate 21, connector fixture 32 can instead be attached to substrate 21, as indicated above, with the connecting terminals of this fixture soldered directly to portions of traces 41 exposed at the upper surface of substrate 21. In this arrangement, connecting cable 31 can then be plugged into connector 32.

Sensing device 20 shown in the side cross section view of FIG. 5B is very similar to the version thereof shown in FIG. 5A except that the pads of the various chips are soldered directly to portions of traces 41 exposed at the upper surface of substrate 21, and so these chips are thereby attached to substrate 21 through employing a flip-chip or surface mount process. In this process, metal pads on the various chips are attached with solder bumps, 43, directly to the exposed portions of traces 41 on substrate 21.

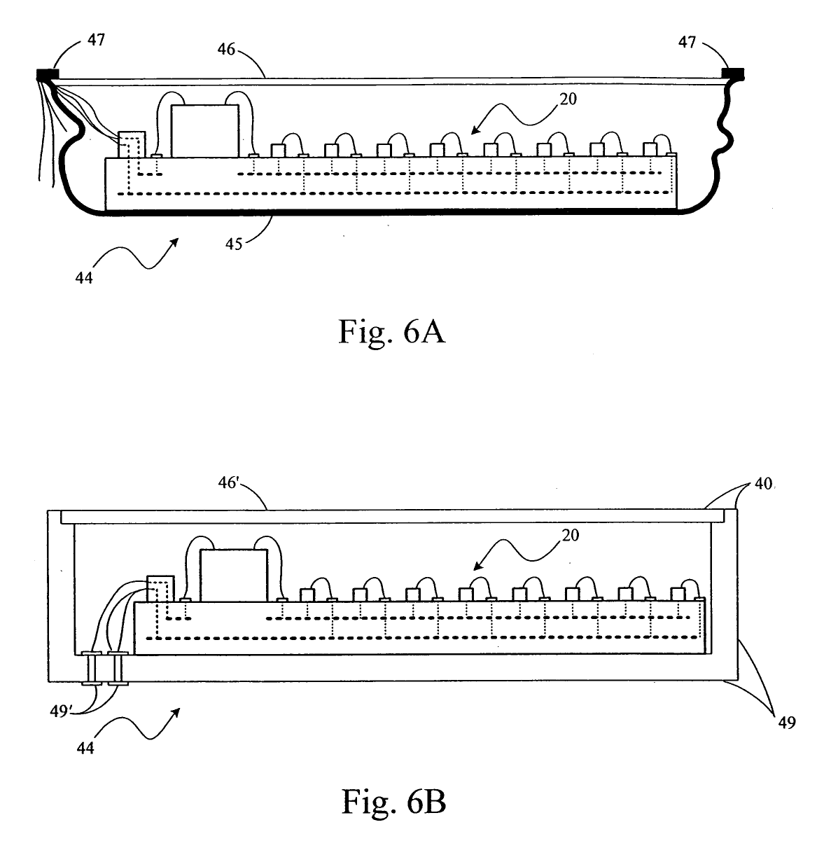

FIG. 6A shows, in a side cross section schematic representation view, the incorporation of sensing device 20 into an adhesive dressing sensing arrangement, 44. The sensor is mounted between a flexible, opaque covering, 45, and a transparent, sterile protective covering, 46, that is against the skin of the wearer during use to thereby form adhesive dressing sensing arrangement 44. An adhesive, 47, is placed around the edges of coverings 45 and 46 to adhere them to one another. Covering 45 can be made of fabric, plastic, or even thin metal, but is to be flexible enough to allow the sensor arrangement to conform to the wearer's body. Transparent covering 46 is also of plastic, but its transparency allows radiation from emitters 26, 27 and 28 to penetrate the wearer's skin and the collection of fluorescent radiation in return by photodetectors 29 and 30 while providing a barrier between the sensor and the skin or body tissues of that wearer. Adhesive 47 also attaches adhesive dressing sensing arrangement 44 to the skin of the patient or subject during times the sensor is being operated but also allows removal when such operation is no longer being conducted.

Another sensing packaging arrangement, 48, perhaps more suitable for long term use, reuse or implantation, utilizes more rigid outer package or housing 40, first indicated above in FIG. 4D, and as seen here in the side cross section schematic representation view of FIG. 6B. Package 40 has an opaque bottom and opaque sides structure, 49, formed of ceramic or plastic materials about an interior recess. Ceramic may be more suitable for implanted devices or hermetic packaging, while plastic is more suitable for external uses (and perhaps some internal uses) and would be lower cost. Package 40 again has a transparent lid, 46′, similar to transparent lid 46 in FIG. 6A, which covers the interior recess in which sensing device 20 is mounted, and lid 46′ is sealed to the sides of structure 49. For hermiticity or compatibility with implantation, glass is preferable for this lid as it can be braised to the ceramic sides. Plastic material for lid 46′ could instead be adhered to sides 49 to provide a lower cost alternative. Electrical contacts for external access to the circuitry in sensing device 1 are provided by including metal feedthroughs 49′ through the bottom of structure 49 in package 40. Although leads from connector 32 are shown attached to these feedthroughs in FIG. 6B, exposed portions of corresponding traces 41 on the upper surface of substrate 21 can instead be attached to these feedthroughs by wirebonding.

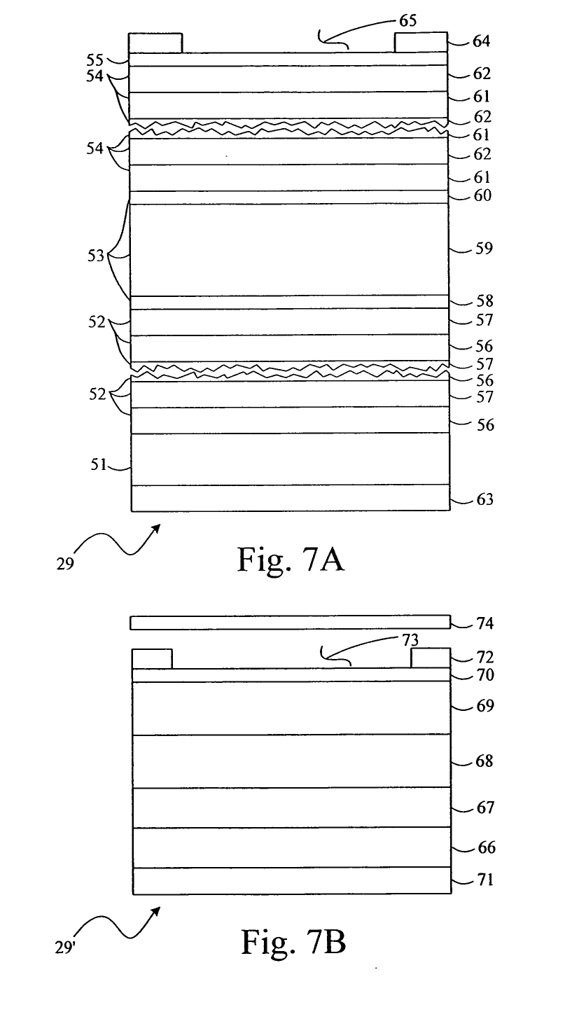

Emitters 26, 27 and 28, and broadband photodetector 30, along with integrated circuit chip 23, can be commercially available devices even though being chosen to have selected wavelengths or wavelength ranges. However, narrowband photodetector 29 needs to be specifically suited to sensing device 20 in having a sufficiently narrow wavelength range of incident electromagnetic radiation sensitivity about the desired center wavelength. A layer diagram for photodetector 29, formed as a resonant cavity photodetector with an internal monolithic filter, is shown in FIG. 7A with the various layers shown there give an indication of structure, but not a true cross section view, in that many dimensions there are exaggerated or reduced relative to one another for purposes of clarity.

The peak quantum efficiency, η, and the full width half maximum (FWHM) Δλ_{1/2} spectral bandwidth of such a detector are given by

$$η = \frac{( 1 - {R}_{i n} ) ( 1 + {R}_{back} {ⅇ}^{- {Γ}_{enh} α d} ) ( 1 - {ⅇ}^{- {Γ}_{enh} α d} )}{{( 1 - \sqrt{{R}_{i n} {R}_{back}} {ⅇ}^{- {Γ}_{enh} α d} )}^{2}}$$

$${Δλ}_{1 / 2} = \frac{λ}{2 π {n}_{λ}} \frac{1 - \sqrt{{R}_{i n} {R}_{back}} {ⅇ}^{- {Γ}_{enh} α d}}{{( {R}_{i n} {R}_{back} )}^{1 / 4} {ⅇ}^{- {Γ}_{enh} α d / 2}}$$

where R_{front} and R_{back} are the input and back mirror reflectivities, respectively, α is the absorption coefficient, λ the resonance wavelength of the detector (which corresponds to the wavelength we desire to detect), d is the total thickness of the absorbing material, n_{λ} the effective cavity length in half or whole multiples of the resonant cavity wavelength, and Γₑₙₕ is the standing wave enhancement factor.

The detector of FIG. 7A is fabricated upon an n⁺-type conductivity GaAs substrate, 51, on which is formed a bottom mirror, 52, with an effective reflectivity of R_{back}, supporting an active region, 53, having a thickness d, which supports in turn a top mirror, 54, with an effective reflectivity Rᵢₙ. A metal contact layer, 55, is provided on top mirror 54. Bottom mirror 52 is formed of a stacked succession of 20 layer pairs (not all shown as indicated by the by the spaced apart broken lines therein) each having a layer of quarter-wavelength thick (i.e. λ/4)) n-type conductivity AlAs, 56, supporting a layer of quarter-wavelength thick (i.e. λ/4)) n-type conductivity AlₓGa₁₋ₓAs, 57. The composition of this last material, AlₓGa₁₋ₓAs, satisfies the constraint of x>0.4.

The layers shown forming active region 53 have thicknesses adding up to 3.5λ. This region has an n-type conductivity AlₓGa₁₋ₓAs layer, 58, with x>0.5, supporting an undoped AlₓGa₁₋ₓAs layer, 59, with x>0.4, that in turn supports a p-type conductivity AlₓGa₁₋ₓAs, As layer, 60, with x>0.5, that together form a p-i-n junction in this region.

Top mirror 54 is about 500 nm thick and formed also as a succession of stacked alternating quarter-wavelength thick layers again not all shown as indicated by the by the spaced apart broken lines therein. These pairs of alternating layers are each formed of a p-type conductivity AlAs layer, 61, and a p-type conductivity AlₓGa₁₋ₓAs layer, 62, with x>0.5.

Top contact layer 55 is p-type conductivity GaAs and is quite thin, i.e. <10 nm. Metal electrode contacts are formed with the contact to the bottom of the device being a bottom area covering contact, 63, and with the top surface being contacted with a patterned contact, 64. An open region, 65, interior to contact 64 is provided to expose the device top surface to thereby serve as a transmission window allowing incident light to enter the detector.

The center wavelength detected by this narrowband detector is determined by the thicknesses of the layers in the mirror regions and the active region described above. The two equations above also indicate that the spectral width of the detecting wavelength range is determined by the effective reflectivities of the two mirrors which are determined by the number of alternating quarter-wavelength layer pairs that are included in each of the mirrors.

This structure results in nearly 100% quantum efficiency and has a bandwidth of 2 nm that is chosen to minimize the wavelength range of incident electromagnetic radiation that would be accepted by the detector for conversion to a corresponding electrical signal representing that radiation. However, clearly, a variety of mirror period and thickness design choices could be made to trade off the quantity of light collected versus the spectral bandwidth.

A layer diagram of a typical p-i-n photodiode, useable as photodetector 29 because of being provided with an external narrowband filter, is shown in FIG. 7B and designated there 29′. In this situation, this detector can have a simpler structure since it need not itself provide the acceptable wavelength range discrimination with respect to incident radiation. This photodetector is fabricated upon an n⁺-type conductivity GaAs substrate, 66, first supporting an n-type conductivity AlₓGa₁₋ₓAs layer, 67, with x>0.5, as the anode on which is provided an unintentionally doped AlₓGa₁₋ₓAs layer more or less intrinsic active region, 68, with x>0.4, that supports thereon a p-type conductivity AlₓGa₁₋ₓAs layer, 69, with x>0.5, as the cathode. A p-type conductivity GaAs contact layer, 70, is provided on cathode layer 69. Metal electrode contacts are formed with the contact to the bottom of the device being a bottom area covering contact, 71, and with the top surface being contacted with a patterned contact, 72. An open region, 73, interior to contact 72 is provided to expose the device top surface to thereby serve as a transmission window allowing incident light to enter the detector. The external filter, 74, indicated above to be used with this photodetector to limit the wavelength range of radiation reaching the photodetector, is positioned above the semiconductor body of detector 29′ in FIG. 7B across from transmission window 73.

The sensing devices of FIGS. 4, 5, 6 and 7 are used to measure the quantity of substances within wearers' bloodstreams by using molecular fluorescence. Such measurements are done in vivo and can be done more or less continuously. These sensing devices first use optical scattering and absorption to locate an appropriate blood vessel beneath the wearer's skin. Then an electromagnetic radiation source emits such radiation to excite the fluorescence followed by the photodetector detecting the fluorescent light.

Making the sensing device operation specific to a measurand of interest requires the fluorescence to be activated by the presence of that measurand. One approach for doing this is to use the FRET process, or the fluorescence resonant energy transfer process, as described above. In this process, two dyes are introduced into the wearer's bloodstream to be conjugated therein with a molecule that reacts preferentially with the measurand of interest. When the reaction occurs, energy is transferred from one dye to the other, and a change in the wavelength of the fluorescent radiation emitted by the latter dye is measured. The operation of these sensing devices in this process therefore requires that dye conjugated molecules enabling such fluorescent measurements to be made be added to the wearer's bloodstream.

Continuous monitoring requires that these sensing devices be worn or used continuously leading to a desire to have such wear and use intrude on the wearer's life as little as possible. These sensing devices use clusters 22 (and 22′) of optoelectronic devices to allow choosing the one thereof which is best “aligned” to a blood vessel (that is, the one of clusters 22—if not using just single cluster 22′—that is providing the “best” backscatter radiation representation signal on a selected basis), and thereby do so inexpensively without restricting the movement of the wearer. This is done dynamically, so that if the sensor moves with respect to its previous location on the wearer's body, the sensing system can choose a new cluster if more optimal for the making of the measurements.

In the embodiment of FIG. 6A where the sensing system is located external to the body of the wearer, it will be attached to the skin of that wearer, most likely on the arm, using adhesive 47 located around the periphery of the sensing system. Alternatively, the sensing system could be attached with a strap around the arm or other limb or extremity. The sensing system could also be a rigid device as in FIG. 6B that is held against the user's body whenever a measurement is to be taken. However, none of these approaches fixes the location of the sensor against the user's arm. Disruptive motions by the user may occur which would cause relative motion between the sensing system and the user's body. By sequentially activating clusters 22 within the sensing system, a cluster can be found which is best aligned to a corresponding blood vessel.

Within a cluster 22 or 22′, first the 660 nm wavelength emitter 26 and the 910 nm wavelength emitter 28 will be caused to emit electromagnetic radiation sequentially, and the intensity of the radiation backscattered from the body of the wearer will be received by narrowband photodetector 29 and broadband photodetector 30. The amount of radiation backscattered is a function of the amount of radiation absorbed (i.e. absorbed radiation is not backscattered.) The absorption and backscattering are a function of wavelength and of type of tissue on which the emitted radiation is incident, and these variations are used to find a blood vessel most simply based on blood in the blood vessel together absorbing more of the incident radiation than do other adjacent tissues. This measurement using broadband photodetector 30 is then repeated by several or all of the other clusters 22 within the sensing system. The differences in the amount of backscattered light registered by each of photodetectors 30 in each of the measuring clusters will be used to determine which cluster is measuring the smallest backscattering radiation intensity typically indicating the cluster that is best aligned to a blood vessel.

The spacing apart of clusters 22 is determined by the size of the blood vessels which vessels are typically 1 mm in diameter. Therefore, a spacing of approximately 0.5 mm between adjacent ones of clusters 22 would make quite likely that at least one cluster will be aligned well with a blood vessel.

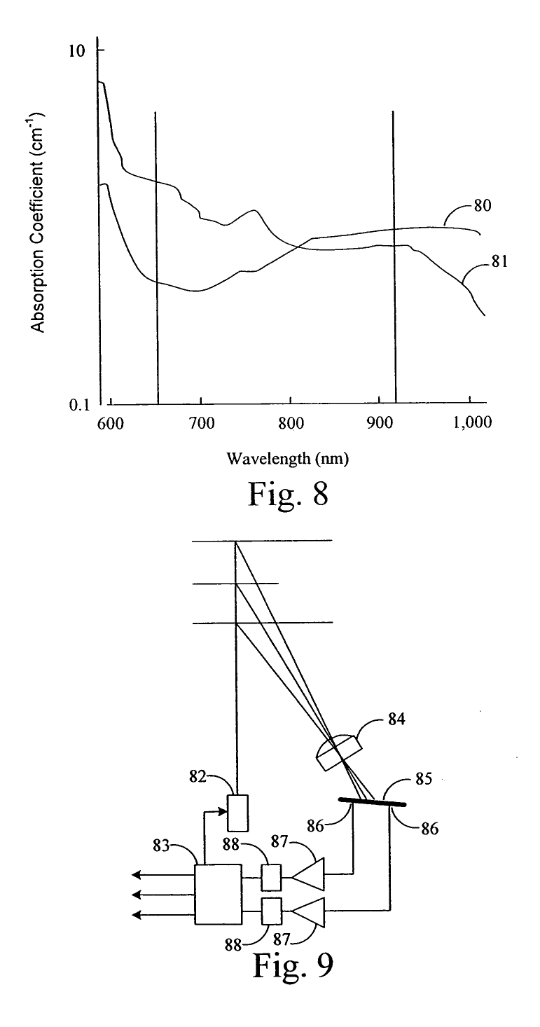

The manner in which this can be done is illustrated in by the graph of FIG. 8 showing a plot, 80, of absorption in oxygentated blood designated HbO₂ and a plot, 81, of absorption in deoxygenated blood designated Hb as a function of wavelength. These absorptions can also be compared to absorption in other tissues shown previously in FIG. 3. The simplest approach is to use radiation at a single wavelength, 660 nm from emitters 26 for instance, and assume that the location with the least amount of backscattered light (corresponding to high absorption in red blood) as measured by photodetectors 30 would correspond to a blood vessel as indicated above.

However, the effects of the degree of oxygenation of the blood, and the thickness of the various tissue layers of the skin, may confound that measurement. Therefore, radiation at two different wavelengths is used sequentially, such as 660 nm and 910 nm, this radiation being emitted from emitters 26 and 28, respectively. A measuring cluster 22 thereby provides the backscattered radiation intensities at these two wavelengths as a basis for determining the ratios of backscattered radiation at those two wavelengths to thereby allow separating out the effects of confounding factors, such blood oxygenation, because of the differing effects of the confounding factors at these two wavelengths. This enables better identification of locations of blood vessels.

While the use of ratios of backscattered radiation at two or more wavelengths is one method, and attractive because of its simplicity, other approaches could also be used. For instance, in arterial blood vessels, the backscattered radiation intensity will be modulated in time due to the wearer's pulse. By monitoring the backscattered radiation versus time, this pulse based modulation of the amplitude of the electrical signal representing the backscattered radiation from the photodetector measuring same will appear if the cluster is sufficiently aligned with a blood vessel.

A third approach applies the well-known principle of “triangulation” as illustrated in the schematic arrangement shown in FIG. 9. In this arrangement, a discontinuity is used that occurs at the surface of the blood vessel between it and adjacent tissues which gives rise to a directly reflected signal. Backscattered radiation suffers multiple scattering events, on average, and therefore has a random angular direction. Directly reflected radiation (that does not suffer any additional scattering) will generally propagate in a specific direction and so apertures, or angle sensitive filters, can be used to accept radiation arriving at the reflection angle. In this arrangement, photodetectors 30, or some of them, could be implemented so as to form a position-sensitive detector or, alternatively, either a charge-coupled device (CCD) or a complementary metal on oxide field-effect transistor (CMOS) array could be added to the assaying system for this purpose.

In the arrangement of FIG. 9, a laser, 82, one of emitters 26, 27 and 28, is directed by a controller, 83, to emit electromagnetic radiation at a slight angle from perpendicular toward the skin surface of the patient, or subject, to be assayed or monitored, and from where it is reflected from the tissues there and the tissues therebelow (schematically shown by three horizontal lines at three possible depth locations from the outer skin surface in the emitter radiation skin impingement area) through a focusing lens, 84, to a position-sensitive detector, 85, shown generally as a bold line in that figure. The emission angle and the geometrical arrangement of the components allow having the reflection due to the skin surface fall outside of detector 85.

Position-sensitive detector 85 receives these reflected radiation signals at an impingement surface thereof and has, on the opposite side surface, split contact electrodes, 86, on opposite sides of this latter surface. The ratios of the currents concurrently measured at each electrode by controller 83, through corresponding ones of amplifiers, 87, and analog-to-digital converters, 88, are a function of the positions of the corresponding reflected optical beams on the impingement surface of the detector between the upward projections of these electrodes.

The location of the reflected beam on the position sensitive detector provides an estimate of the depth of the source of the reflection as can be seen from the geometry in FIG. 9, which in turn helps to distinguish reflection at the surface of a blood vessel from other causes of reflection. If instead of this analog signal position-sensitive detector arrangement, a CCD or CMOS array is used in place of detector 85, electrodes 86, amplifiers 87 and converters 88, the pixel locations in the array therein receiving the maximum optical signal are determined by controller 83 from the periodic sequential pixel data transfers thereto from that array. This determination allows controller 83 to further determine the location (angle) of the reflected beam as the basis for, in turn, estimating the corresponding depth of the reflection location below the skin outer surface. Again, use of radiation at two different wavelengths will assist in providing a more unambiguous determination.

After determination of which of clusters 22 is best aligned to a blood vessel, this cluster is used for a fluorescence measurement of an element of the bloodstream therein. However, the preceding blood vessel location procedure can be repeated at regular time intervals to check for possible movement of the sensing system to a more poorly aligned position, and subsequent fluorescence measurements can be shifted to a different cluster that becomes better aligned with a blood vessel as a result of the sensing system movement.

Enabling a fluorescence measurement requires that a molecular probe comprising the dye conjugated antibody or enzyme with two fluorphores attached thereto be added to the bloodstream of the wearer of the sensing system which is typically done when this system is positioned on the wearer. This probe addition can be accomplished, for example, by the use of a transdermal patch from which the dye conjugated molecules are transferred across the skin rather than by injection. This has the advantage of allowing the patient to be untethered, eliminates a painful injection and will allow a continuous addition of the probe molecules into the bloodstream. However, alternatives include a one time injection of the molecular probe into the bloodstream of the wearer, or the use of an IV (intravenous device) that allows for a controlled addition of the molecular probe to the bloodstream of the wearer.

Use of an implantable version of the sensing device such as that shown in FIG. 6B allows use of all of these alternatives for introducing the molecular probe into the bloodstream of the person in which the device is implanted. In addition, however, the source of the molecular probe can be included in the implanted device. This molecular probe would from there be slowly released into that person's bloodstream by incorporating it in a drug delivery device similar to a drug-eluting stent for example.

The molecular probe, as indicated above, has dyes conjugated to an antibody or an enzyme. In the situation that the measurand is a protein in the subject's bloodstream, such as the cardiac proteins described above for heart attack patients, then such dyes can be conjugated to either antibodies or enzymes. As a specific example, in the case of the cardiac proteins, the fluorophores can be conjugated with troponin antibodies Protein G or Protein A complexes, as examples. In the measurement of chemical substances in the subject's bloodstream, like glucose or creatinine, an enzyme conjugated with a fluorophore is more appropriate.

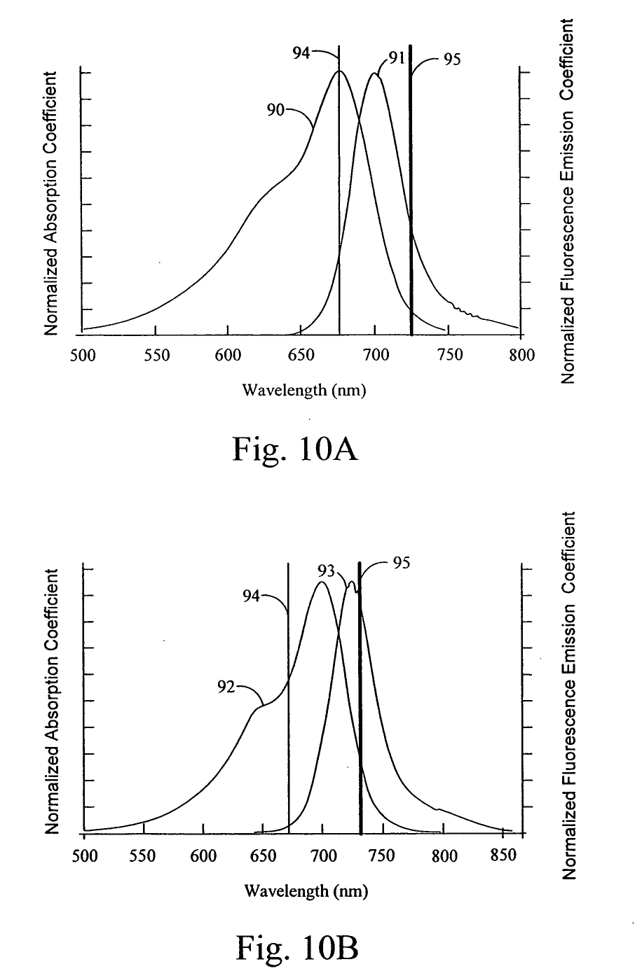

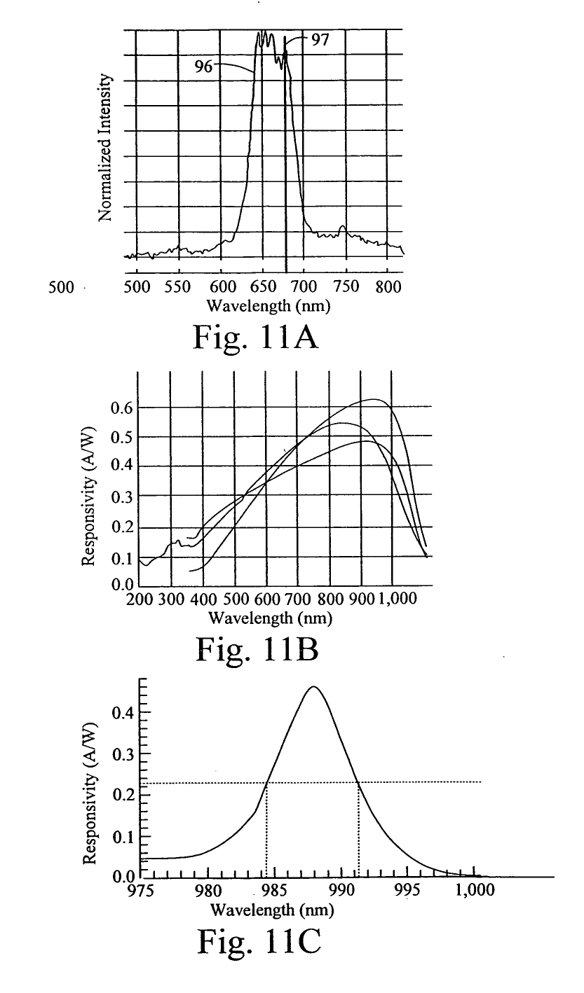

Once the molecular probe is circulating within the bloodstream of the sensing system wearer, and the best aligned one of clusters 22 has been identified as described above, the fluorescence measurement can be made. The graphs of FIG. 10 illustrates the considerations in the design of the sensing system including the electromagnetic radiation emitting source, the two fluorphore dyes chosen for use, and the fluorescent radiation photodetector. The absorption and emission spectra, 90 and 91, of the commercially available dye Alexa Fluor 680 are shown in FIG. 10A, and the absorption and emission spectra, 92 and 93, of the commercially available dye Alexa Fluor 700 are shown in FIG. 10B. From the FRET process description above in connection with FIG. 2, one of the requirements for suitable dyes is that the emission peak of the first dye (Alexa Fluor 680) overlaps with the absorption peak of the second dye (Alexa Fluor 700). This combination satisfies that condition. Note also, that the absorption and emission peaks of these dyes falls within the radiation wavelength transmissibility window corresponding to the transparency of bodily tissues, as indicated in FIG. 3, which allows the exciting beams from emitter 27 to penetrate more deeply into the bodily tissues of the wearer, and also allows the fluorescence emission beam to escape from body tissues. Finally, the emission spectrum, 94, of emitter 27 chosen to be a VCSEL and the response spectrum, 95, of narrowband photodetector 29 are shown in both FIGS. 10A and 10B

Electromagnetic radiation from emitter 27 with the appropriate wavelength, i.e. 680 nm, is absorbed by the first dye, i.e. Alexa Fluor 680. If the molecular probe has not attached to an antigen or measurand of interest, the radiation emitted therefrom will be peaked at the emission peak of that of dye 1, i.e. at about 700 nm. However, if it has reacted through binding to an antigen, then the energy absorbed by dye 1 will be transferred nonradiatively to, and absorbed by, dye 2 as indicated for the FRET process described above. Emission by fluorescence will now occur from dye 2 peaked at the emission peak of that of dye, i.e. at a wavelength of approximately 730=m.

The advantage of the use of a VCSEL as the emitter, and the use of a narrowband photodetector, are illustrated in FIG. 11. The plots in these graphs illustrate, first in FIG. 11A, the spectrum of a LED emission, 96, versus that of a VCSEL emission, 97, and so the relative emission widths thereof of 50 nm and 1 nm, respectively. FIG. 11B is a graph with the plots of the spectral responsivities of various common silicon photodetectors. The relatively large responsive width can be compared to the much narrower spectral responsivity shown in FIG. 11C of a detector fabricated with, in effect, a monolithically integrated filter for the detector device using repeated paired layers mirrors similar to the structure represented in FIG. 7A, and which would be similar to the spectral widths achievable with the photodetectors of FIG. 11B used with an external filter as represented in FIG. 7B. The ability to restrict the ranges of wavelengths of emitted and detected radiation results in better signal-to-noise ratios in the measuring system. For instance, radiation emitted from the emitter 27 used to excite the dye fluorescence isn't confused with the resulting fluorescent radiation emissions as result of using such narrowband detectors.

A straightforward intensity measurement as described above is preferred for reasons of simplicity. However, especially when one is attempting to measure very low concentrations of a measurand, other techniques may necessary to improve the signal-to-noise ratios. For example, the emitter may be pulsed with a selected frequency and the detector can be phase-locked to that frequency as a basis for eliminating noise at other frequencies. Radiation intensities that are receive that do not vary with that selected frequency are ignored.

In addition to having a particular wavelength, fluorescent radiation emissions usually have a characteristic decay time that is a function of the dye chosen for use and the molecules to which it becomes attached. In addition to using wavelength, the decay time can be measured to distinguish such fluorescent radiation emissions from reacted molecular probes versus the unreacted molecular probes.

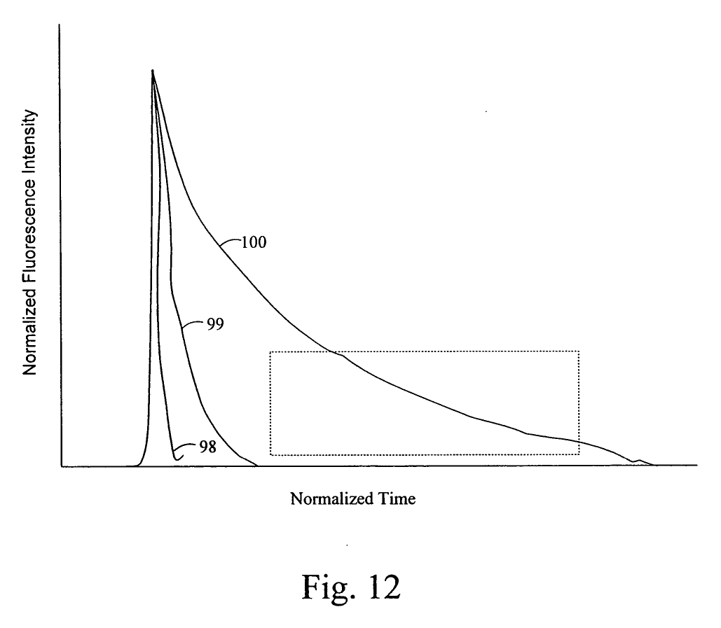

The emitter can be pulsed with a short duration pulse and then the signal from the photodetector representing fluorescent radiation is obtained gated in time to be correlated with those pulses so as not to confuse the excitation radiation with the fluorescent radiation. This is illustrated in FIG. 12 beginning with the emitter pulse from which Raman and Rayleigh light scattering results, 98, against the background fluorescense, 99, followed by the dye fluorescence decay, 100. This dye fluorescence decays over a sufficiently longer time to allow selecting a time of measurement interval shown in the dashed line rectangle that occurs after the other unwanted output radiation components have decayed.

In all three of these measurement output signal acquisition variations, one of the advantages of the VCSEL over a LED or an edge-emitting laser as excitation emitters in clusters 22 is the speed of the device. The VCSEL can emit pulses that are a tenth of the duration of pulses emitted by an edge-emitting laser, and a hundredth of the duration of pulses emitted by a LED.

System control and signal processing of system signals will be carried out by the control and signal processing capabilities provided in integrated circuit chip 23 that is incorporated in the sensing device system. The processor of this chip will manage the process of optimally choosing which of optoelectronic clusters 22 is to be used for subsequent fluorescent radiation measurements, by directing electrical current to the appropriate ones of the other devices in the sensing device system at the predetermined time intervals, and by processing the electrical signals received from the photodetectors representing the backscattered and fluorescent radiations sensed by them. Signal processing to identify the optimal one of clusters 22 for subsequent fluorescent radiation emissions measurements includes calculating the ratios of the received photodetector electrical signals representing the backscattered radiations of the two wavelengths of electromagnetic radiation emitted within each cluster, and comparing the resulting ratios from one cluster to another. Depending upon the measurement approaches used, such signal processing will also determine the pulse rate of the wearer by monitoring the change in those received photodetector signals and ratios thereof over time.

Once an optimal sensing device system optoelectronic cluster 22 is chosen, the processor of chip 23 will direct electrical current to the radiation emitter in that cluster to thereby excite the fluorescence, and monitor the electrical signal received from narrowband photodetector 29 representing the dye fluorescent radiation emissions. Signal processing monitoring functions will include receiving and recording the photodetector electrical signals representing fluorescent radiation emissions as a function of time. If a phased-locked loop approach is used, the signal processing will reject signals without the proper frequency characteristics. If time resolved techniques are used, short pulses will be directed to be generated, and the response of the photodetector versus time will be recorded. If a triangulation approach is used, the location of the reflecting discontinuity below the surface will be estimated to determine the likelihood that it is a blood vessel. If a separate narrowband detector is used for the emissions of the two dyes in a FRET assay, then all of the signal processing will be carried out as a function of the ratios of the two signals or of the time constants thereof or both. The output of the signal processing will be an estimate of the concentration of the measurand in the wearer's bloodstream based on the measurement data and its variation over time.

Reducing the cost of, and keeping small the size of, the sensor can be accomplished to a degree by limiting the amount of signal processing carried out in chip 23. Although it must retain its control functions such as directing electrical currents to the emitters in optoelectronic clusters 22, and it must provide sufficient signal processing to record the backscattering related output signals from the photodetectors and determine the appropriate one of clusters 22 to use, little else is required to be accomplished by the processor of that chip. Data recorded from the fluorescent radiation emissions measurements can be transmitted from the sensing device system to another remote signal processor. This could be done via an electrical cable or by telemetry. Although an electrical cable can be used to provide electrical power to the sensing device system and to acquire data, telemetry can be used instead with an electrical power source provided in the sensing device system. This configuration is preferable in enabling the patient to remain untethered, or minimizing any other related restrictions.

Although the present invention has been described with reference to preferred embodiments, workers skilled in the art will recognize that changes may be made in form and detail without departing from the spirit and scope of the invention.

1. A method of in vivo assaying for a presence of selected constituents in bloodstreams in circulatory system passageways in mammalian bodies having relatively small electromagnetic radiation absorption in at least one transmission wavelength range for transmissions of electromagnetic radiation between such passageways and outer surfaces of corresponding bodily skin, said method comprising:
introducing in a said bloodstream a probe comprising a binding base capable of binding to at least one of said selected constituents and of also concurrently conjugating to two different fluorophores of which one has an emission spectra peak distribution in said transmission wavelength range that overlaps an absorption spectra peak distribution of that one remaining,
radiating at least some of said passageways, thereby forming evaluative radiated passageways, from at least one location on said outer surfaces of said corresponding skin with evaluation electromagnetic radiation of wavelengths in said transmission wavelength range and also in an absorption spectra range of that said fluorophore having an overlapping emission spectra peak distribution, and
detecting electromagnetic radiation at least at one location on said outer surfaces of said corresponding skin that has been transmitted from said evaluative radiated passageways of wavelengths in said transmission wavelength range and also in an emission spectra range of that said fluorophore having an overlapping absorption spectra peak distribution wherein said emission spectra range differs from said overlapping emission spectra peak distribution.

2. The method of claim 1 further comprising radiating at least some of said passageways, thereby forming survey radiated passageways, with survey electromagnetic radiation of wavelengths in said transmission wavelength range, detecting electromagnetic radiation transmitted from said survey radiated passageways of wavelengths in said transmission wavelength range at plural locations on said outer surfaces of said corresponding skin, and determining that one of said plural locations having a smallest intensity of backscattered electromagnetic radiation relative to those remaining.

3. The method of claim 1 further comprising radiating at least some of said passageways, thereby forming survey radiated passageways, with survey electromagnetic radiation of wavelengths in said transmission wavelength range, detecting electromagnetic radiation transmitted from said survey radiated passageways of wavelengths in said transmission wavelength range using a photodetector having a detecting surface on which said electromagnetic radiation from said survey radiated containing structures impinges, and determining that location on said detecting surface having a largest intensity of impinging electromagnetic radiation.

4. The method of claim 1 wherein said introducing in said bloodstreams of said probe is accomplished through an injector extending through said outer surfaces of corresponding bodily skin into said circulatory system passageways.

5. The method of claim 1 wherein said introducing in said bloodstreams of said probe is accomplished through a source of said probe being positioned against said outer surfaces of corresponding bodily skin to diffuse said probe into said circulatory system passageways.

6. The method of claim 1 wherein said binding base is selected from antibodies and enzymes.

7. The method of claim 1 wherein said radiating of said evaluative radiated passageways with electromagnetic radiation is accomplished through radiating pulses of said electromagnetic radiation at a selected frequency.

8. The method of claim 1 wherein said radiating of said evaluative radiated passageways with electromagnetic radiation is accomplished through radiating electromagnetic radiation of a first selected wavelength and thereafter by radiating electromagnetic radiation of a second selected wavelength.

9. The method of claim 1 wherein said detecting of electromagnetic radiation from said evaluative radiated passageways is accomplished through determining frequencies occurring in changes in intensity of said electromagnetic radiation.

10. The method of claim 1 wherein said detecting of electromagnetic radiation from said evaluative radiated passageways is accomplished through determining of intensity of only a later portion of said electromagnetic radiation that has been detected.

11. The method of claim 1 wherein said electromagnetic radiation detected from said evaluative radiated passageways provides information that is incorporated into signals transmitted to a remote receiver.

12. The method of claim 2 wherein said radiating of said evaluative radiated passageways with electromagnetic radiation is accomplished through radiating electromagnetic radiation of a first selected wavelength and thereafter by radiating electromagnetic radiation of a second selected wavelength.

13. The method of claim 2 wherein said detecting of electromagnetic radiation from said evaluative radiated passageways is accomplished through determining frequencies occurring in changes in intensity of said electromagnetic radiation.

14. The method of claim 3 wherein said radiating of said evaluative radiated passageways with electromagnetic radiation is accomplished through radiating electromagnetic radiation of a first selected wavelength and thereafter by radiating electromagnetic radiation of a second selected wavelength.

15. A method of assaying for a presence of selected constituents in liquid mixtures confined by corresponding containing structures having relatively small electromagnetic radiation absorption in at least one transmission wavelength range for transmissions of electromagnetic radiation therethrough, said method comprising:
introducing in said liquid mixture a probe comprising a binding base capable of binding to at least one of said selected constituents and of also concurrently conjugating to two different fluorophores of which one has an emission spectra peak distribution in said transmission wavelength range that overlaps an absorption spectra peak distribution of that one remaining,
radiating at least some of said containing structures, thereby forming evaluative radiated containing structures, with evaluation electromagnetic radiation of wavelengths in said transmission wavelength range and also in an absorption spectra range of that said fluorophore having an overlapping emission spectra peak distribution, and
detecting electromagnetic radiation that has been transmitted from said evaluative radiated containing structures of wavelengths in said transmission wavelength range and also in an emission spectra range of that said fluorophore having an overlapping absorption spectra peak distribution wherein said emission spectra range differs from said overlapping emission spectra peak distribution.

16. The method of claim 15 further comprising radiating at least some of said containing structures, thereby forming survey radiated containing structures, with survey electromagnetic radiation of wavelengths in said transmission wavelength range, detecting electromagnetic radiation from said survey containing structures of wavelengths in said transmission wavelength range at plural locations across from said surveyed radiated containing structures, and determining that one of said plural locations having an extreme intensity of backscattered electromagnetic radiation relative to those remaining.

17. The method of claim 15 further comprising radiating at least some of said containing structures, thereby forming survey radiated containing structures, with survey electromagnetic radiation of wavelengths in said transmission wavelength range, detecting electromagnetic radiation from said survey containing structures of wavelengths in said transmission wavelength range using a photodetector having a detecting surface on which said electromagnetic radiation from said survey radiated containing structures impinges, and determining that location on said detecting surface having a largest intensity of impinging electromagnetic radiation.

18. A body supported bloodstream biomarker evaluation system for assaying for a presence of selected constituents in bloodstreams in circulatory system passageways in mammalian bodies having relatively small electromagnetic absorption in at least one transmission wavelength range between such passageways and outer surfaces of corresponding bodily skin in which bloodstreams a probe has been introduced comprising a binding base capable of binding to at least one of said selected constituents and of also concurrently conjugating to two different fluorophores of which one has an emission spectra peak distribution in said transmission wavelength range that overlaps an absorption spectra peak distribution of that one remaining, said system comprising:
a substrate,
a first electromagnetic radiation emitter supported by said substrate and capable of emitting evaluation electromagnetic radiation of wavelengths in said transmission wavelength range and also capable of emitting evaluation electromagnetic radiation of wavelengths in an absorption spectra range of that said fluorophore having an overlapping emission spectra peak distribution; and
a first electromagnetic radiation detector supported by said substrate and capable of detecting electromagnetic radiation of wavelengths in said transmission wavelength range and also in an emission spectra range of that said fluorophore having an overlapping absorption spectra peak distribution wherein said emission spectra range differs from said overlapping emission spectra peak distribution.

19. The system of claim 18 further comprising a housing about an interior space therein bounded at least in part by a window side of said housing that is transparent to electromagnetic radiation of wavelengths in said transmission wavelength range, said housing having said substrate therein with said emitter and said detector in said interior space across from said window side.

20. The system of claim 18 further comprising a plurality of electromagnetic radiation emitters, including said first electromagnetic radiation emitter, each supported by said substrate and capable of emitting electromagnetic radiation of wavelengths in said transmission wavelength range, and a plurality of electromagnetic radiation detectors, including said first electromagnetic radiation detector, each supported by said substrate and capable of detecting electromagnetic radiation of wavelengths in said transmission wavelength range.

21. The system of claim 18 further comprising a communications integrated circuit supported by said substrate which is capable of receiving signals related to electromagnetic radiation detected by said first electromagnetic radiation detector and transmitting representations thereof to a remote receiving device.

22. The system of claim 20 wherein members from said plurality of electromagnetic radiation emitters and members from plurality of electromagnetic radiation detectors are located relatively near one another to form survey and evaluation groups and a plurality of said survey and evaluation groups are each positioned at a locations over said substrate differing from one another.
